(19) United States
(12) Patent Application Publication
(10) Pub. No.: US 2015/0283300 A1
(43) Pub. Date: Oct. 8, 2015

(54) BIOACTIVE GLASSES WITH SURFACE IMMOBILIZED PEPTIDES AND USES THEREOF

(71) Applicants: Gregory J. Pomrink, Newberry, FL (US); Cecilia A. Cao, Gainesville, FL (US); Zehra Tosun, Gainesvile, FL (US); Layne Howell, Gainesville, FL (US)

(72) Inventors: Gregory J. Pomrink, Newberry, FL (US); Cecilia A. Cao, Gainesville, FL (US); Zehra Tosun, Gainesvile, FL (US); Layne Howell, Gainesville, FL (US)

(21) Appl. No.: 14/504,956

(22) Filed: Oct. 2, 2014

Related U.S. Application Data
(60) Provisional application No. 61/974,818, filed on Apr. 3, 2014.

(51) Int. Cl. A61L 27/28; A61L 27/10; A61L 27/56; A61L 27/54

(52) U.S. Cl. CPC A61L 27/28; A61L 27/54; A61L 27/10; A61L 27/56; A61L 2430/02; A61L 2420/02

(57) ABSTRACT

The invention relates to bioactive glass compositions that include bioactive glass with surface immobilized peptides and methods and uses thereof.

### RELATED APPLICATIONS

The present patent document claims the benefit of the filing date under 35 U.S.C. §119(e) of Provisional U.S. Patent Application Ser. No. 61/974,818 filed Apr. 3, 2014, content of which is hereby incorporated by reference.

### BACKGROUND

Bioactive glass was originally developed in 1969 by L. Hench. Bioactive glasses were developed that serve as bone replacement materials, with studies showing that bioactive glass can induce or aid in osteogenesis (Hench et al., J. Biomed. Mater. Res. 5:117-141 (1971)). Bioactive glass can form strong and stable bonds with bone (Piotrowski et al., J. Biomed. Mater. Res. 9:47-61(1975)). Further, bioactive glass is not considered toxic to bone or soft tissue (Wilson et al., J. Biomed. Mater. Res. 805-817 (1981)). Exemplary bioactive glasses known in the art include 45S5, 45S5B1, 58S, and 570C30. The original bioactive glass, 45S5, is melt-derived. Sol-gel derived glasses have nanopores that allow for increased surface area and bioactivity.

Various efforts have been made to improve the bioactivity of bioactive glasses using various surface coatings. For example, in an article entitled “Surface functionalization of Bioglass-derived porous scaffolds”, Boccaccini et al. describe bioactive glass modified by applying 3-aminopropyl-triethoxysilane (Acta Biomaterials 3, pp. 551-562 (2007)). Boccaccini et al. describes modifying 45S5 Bioglass-based glass-ceramic scaffolds using organic solvents and concludes that the surface-functionalized scaffolds are ready for protein immobilization and can be used for protein release studies or to fabricate Bioglass-protein hybrids.

Other examples include Ammar “The Influence of Peptide Modification of Bioactive Glass on Human Mesenchymal Stem Cell Growth and function,” Thesis and Dissertations, Paper 1355, Lehigh University (2011). Ammar writes that Bioactive glass is a material that has high bioactivity and can induce bone formation in bone progenitor cells but studies have shown that it has no effect on human mesenchymal stem cells (hMSCs). Ammar hypothesized that the potentials of the bioactive glass can be broadened to include the differentiation of hMSCs by the incorporation of peptides from proteins known for their ability to induce differentiation of hMSCs into bone cells. For that, three peptides sequences that contain domains from fibronectin, BMP-2 and BMP-9 proteins that are known to promote adhesion, differentiation and osteogenesis in hMSCs were selected and synthesized for their studies.

U.S. Pat. No. 8,367,602 to Lyngstadaas et al. (the '602 patent) describes artificial peptides optimized for the induction and/or stimulation of mineralization and/or biomineralization in vivo and in vitro. The '602 patent further describes administering to a cell culture, tissue, surface, and/or solution a pharmaceutical composition that includes a peptide, where the surface may be a metal, metal oxide, metal hydroxide, metal hydride, hydroxyl apatite, aragonite, bioglass, glass, polyurethane, polymeric medical prosthetic device, medical prosthetic device, biological surface, and combinations thereof. The '602 patent further describes a process for mineral precipitation and/or biomineralization inducing and/or stimulating surface. The '602 patent also describes a method where a surface to be mineralized is contacted with a peptide to provide the peptide on the surface.

U.S. Pat. No. 6,413,538 to Garcia, et. al. (the '538 patent) describes a bioactive glass or ceramic substrate having bound cell adhesion molecules. The '538 patent relates to the synthesis of bioactive ceramic templates for optimum in vitro formation of bone and bone-like tissue, and the use of bioactive substrates for the enhanced cellular attachment and function of anchorage-dependent cells. The '538 patent describes a bioactive glass or ceramic material substrate for anchorage-dependent cells that has been treated prior to contact with the cells by immersion in a first aqueous solution containing ions in a concentration typical of interstitial fluid followed by immersion in a second aqueous solution consisting essentially of at least one cell adhesion molecule, under conditions effective for achieving greater cellular attachment strength of the anchorage-dependent cells. The '538 patent further describes the formation of an implant that includes an implant material for treating defects in sites where tissue is made by anchorage-dependent cells, the implant material being coated with a substrate of bioactive glass or ceramic material for anchorage-dependent cells that has been treated prior to contact with the cells by immersion in a first aqueous solution containing ions typical of interstitial fluid followed by immersion in a second aqueous solution consisting essentially of at least one cell adhesion molecule, under conditions effective for achieving greater cellular attachment strength of the anchorage-dependent cells. Cell adhesion molecules disclosed in the '538 patent include fibronectin, vitronectin, laminin, collagen, osteopontin, bone sialoprotein, thrombospondin, and fibrinogen, and combinations thereof.

Although some of the bioactiove glass compositions as well as methods previously described may be adequate as bone replacement materials, improved methods and compositions are desirable.

### SUMMARY OF THE INVENTION

Provided are bioactive glasses with surface immobilized peptides wherein the peptides are bone resorption inhibitors such as WP9QY(W9), OP3-4, or RANKL inhibitor peptide and mixtures thereof.

Also provided are bioactive glasses with surface immobilized peptides wherein the peptides are bone formulation stimulators such as B2A, P1, P2, P3, P4, P24, P15, TP508, OGP, or PTH and mixtures thereof.

Further provided are bioactive glasses with surface immobilized peptides wherein the peptides are both bone resorption inhibitors and bone formation stimulators such as NBD, CCGRP, or W9 and mixtures thereof.

Further provided are bioactive glasses with surface immobilized peptides wherein the peptides are bone targeting peptides such as (Asp)₆, (Asp)₈, or (Asp, Ser, Ser)₆ and mixtures thereof.

In the above bioactive compositions, the bioglass may be in a granular form, particulate form, matt form, fiber form, hemostatic sponge form, foam form, paste or putty form, or sphere or bead form, or a combination thereof. The bioglass may be 45S5 bioglass, 45S5B1, 58S, and/or S70030. The bioglass may be porous. The bioactive compositions may further include at least one therapeutic agent selected from the group consisting of antimicrobials, antibiotics, collagen, fibrin, fibronectin, Vitamin E, and would repair dressing. The bioactive composition may be for filling bone defects, gaps in bone or gaps in skeletal system of a subject. When the composition is placed in contact with a bone at or near a site of a bone defect, the composition is capable of eliminating the adsorption of proteins that would result in the adhesion of unspecific cells leading to fibrous integration, enhancing the specific attachment of osteogenic cells for the establishment of a tight bone-implant interface; and providing integrin-mediated signals for provoking bone healing mechanisms. In the compositions, the peptides may be immobilized on the bioglass by process of plasma modification, silanation, biotinylation, or layer by layer coating assembly.

Further provided is a bioactive glass composition for filling bone defects, gaps in bone or gaps in skeletal system of a subject. The composition, when placed in contact with a bone at or near a site of a bone defect, is capable of eliminating the adsorption of proteins that would result in the adhesion of unspecific cells leading to fibrous integration, enhancing the specific attachment of osteogenic cells for the establishment of a tight bone-implant interface; and providing integrin-mediated signals for provoking bone healing mechanisms.

Further provided is a method for treating a bone having a bone defect comprising contacting the bone at or near the site of the bone defect with any of the above-described bioactive glasses with surface immobilized peptides. In the method, the peptides may be WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

Further provided is a method for making bioactive glass coated with surface immobilized peptides. The method includes dissolving one or more peptides, diluting the dissolved one or more peptides, and contacting a bioactive glass with the dissolved peptides to adsorb the one or more peptides on the surface of the bioactive glass, wherein the one or more peptides bind free —OH groups on a surface of the bioactive glass. In the method, the peptides may be WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

Further provided is a method of treating a bone having a bone defect. The method includes contacting the bone at or near the site of the bone defect with the bioactive glass compositions described herein.

Further provided is a method for making bioactive glass coated with surface immobilized peptides. The method includes dissolving one or more peptides, diluting the dissolved one or more peptides, and contacting a bioactive glass with the dissolved peptides to adsorb the one or more peptides on the surface of the bioactive glass, wherein the one or more peptides bind free —OH groups on a surface of the bioactive glass. In the method, the peptides may be WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

Further provided is a method for making bioactive glass coated with surface immobilized peptides. The method includes comprising biotinylating the c-terminus end of one or more peptides, coating a bioactive glass with the one or more biotinylated peptides, blocking the coated bioactive glass, and incubating the blocked bioactive glass. In the method, the peptides may be WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

Further provided is a method for making bioactive glass coated with surface immobilized peptides. The method includes silanating one or more peptides, coating a bioactive glass with the one or more biotinylated peptides, blocking the coated bioactive glass, and incubating the blocked bioactive glass. In the method, the silanating step may include contacting the one or more peptides with, e.g., 4-aminobutyltriethoxysilane. In the method, the peptides may be WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

Further provided is a method for promoting bone remodeling in a subject including contacting the bone in need of bone remodeling with any of the bioactive glass compositions described herein.

Also provided is a putty composition, which comprises a bioactive glass with surface immobilized peptides, glycerin, and polyethylene glycol. The peptides may be WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

Also provided is an implant with a coating of bioactive glass with surface immobilized peptides. The peptides may be WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

### DETAILED DESCRIPTION OF THE INVENTION

Bioglass and other bioactive ceramics have an excellent bone-bonding capability due to their ability to deposit hydroxyapatite, which has a high capacity to bind proteins. However, the bioactive function of bioactive glasses can be hampered by crystallization. Therefore, surface functionalization is necessary to maintain the protein binding ability of partially crystallized silicate systems, e.g. bioglass derived glass ceramic scaffolds.

The present invention discloses the preparation and immobilization of bioactive molecules, peptides, proteins and others on bioactive glasses and compositions provided with surface immobilized molecules, peptides, proteins and others.

The glass ceramic component is initially functionalized. Various methods of preparing functionalized bioglass are knows and include, e.g., plasma modification, silanation, biotinylation, layer by layer coating assembly, and bioactive glasses' naturally occurring ion exchange mechanism or other available methods.

Once bioglass is functionalized, the molecules, peptides, proteins and others may be immobilized. Preferably, the molecules, peptides, proteins and others are immobilized prior to glass ceramic component absorbing blood or body fluid undergoes an ion exchange with the surrounding body fluid.

Once the bioglass is placed in contact with the body, bioglass releases calcium, phosphate and boron ions, which activate a genetic cascade responsible for osteoblast proliferation and differentiation and, subsequently, promotes the increased rate regeneration of hard tissue. In addition, the glass ceramic of the present invention provides specific mediated signals through the bioactive molecule immobilization to promote angiogenesis and facilitates wound healing, and angiogenesis along with the differentiation and proliferation of osteoblasts (defined as osteostimulation), which increases the rate of regeneration of hard tissue.

The main idea behind these methodologies is as follows: (1) to eliminate the adsorption of proteins that would result in the adhesion of unspecific cells leading to fibrous integration; (2) to enhance the specific attachment of osteogenic cells for the establishment of a tight bone-implant interface; (3) to provide integrin-mediated signals for provoking the bone healing mechanisms.

Bioactive glass used in the invention may be melt-derived or sol-gel derived.

In certain embodiments, depending on their composition, bioactive glasses of the invention may bind to soft tissues, hard tissues, or both soft and hard tissues.

The composition of the bioactive glass may be adjusted to modulate the degree of bioactivity. For example, the bioactive glass may be pre-treated with TRIS buffer. See, e.g., U.S. application Ser. No. 13/039,627, filed May 3, 2011, which is incorporated herein in its entirety.

Furthermore, in certain embodiments, borate may be added to bioactive glass to control the rate of degradation. See, e.g., U.S. Provisional Application Ser. No. 61/782,728, filed Mar. 14, 2013, which is incorporated herein in its entirety.

In certain other embodiments, additional elements, such as copper, zinc, and strontium may be added to bioactive glass to facilitate healthy bone growth.

Bioactive glass that may also be suitable include glasses having about 40 to about 60 wt % SiO₂, about 10 to about 34 wt % Na₂O, up to about 20 wt % K₂O, up to about 5 wt % MgO, about 10 to about 35 wt % CaO, 0 to about 35 wt % SrO, up to about 20 wt % B₂O₃, and/or about 0.5 to about 12 wt % P₂O₅. In certain embodiments, the bioactive glass may additionally contain up to 10 wt % CaF₂.

Bioactive glass particles, fibers, meshes or sheets may be prepared by a sol-gel method. Methods of preparing such bioactive active glasses are described in Pereira, M. et al., “Bioactive glass and hybrid scaffolds prepared by sol-gel method for bone tissue engineering” (Advances in Applied Ceramics, 2005, 104(1): 35-42) and in Chen, Q. et al. (Chen et al., “A new sol-gel process for producing Na₂O-containing bioactive glass ceramics” Acta Biomaterialia, 2010, 6(10):4143-4153).

The composition can be allowed to solidify. In some embodiments, particles of bioactive glass may be sintered to form a porous glass.

Repeated cooling and reheating may be performed on the solidified or sintered bioactive glass, with or without spinning, to draw the bioactive glass produced into fibers. A glass drawing apparatus may be coupled to the spinner and the source of molten bioactive glass, such as molten bioactive glass present in a crucible, for the formation of bioactive glass fibers. The individual fibers can then be joined to one another, such as by use of an adhesive, to form a mesh. Alternatively, the bioactive glass in molten form may be placed in a cast or mold to form a sheet or another desired shape.

The bioactive glass particles, fibers, meshes or sheets may further comprise any one or more of adhesives, grafted bone tissue, in vitro-generated bone tissue, collagen, calcium phosphate, stabilizers, antibiotics, antibacterial agents, antimicrobials, drugs, pigments, X-ray contrast media, fillers, and other materials that facilitate grafting of bioactive glass to bone.

A bioactive glass ceramic material suitable for the present compositions and methods may have silica, sodium, calcium, strontium, phosphorous, and boron present, as well as combinations thereof. In some embodiments, sodium, boron, strontium, and calcium may each be present in the compositions in an amount of about 1% to about 99%, based on the weight of the bioactive glass ceramic. In further embodiments, sodium, boron, strontium and calcium may each be present in the composition in about 1%, about 2%, about 3%, about 4%, about 5%, about 6%, about 7%, about 8%, about 9%, or about 10%. In certain embodiments, silica, sodium, boron, and calcium may each be present in the composition in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, strontium, and boron with only traces of the other(s). The term “about” as it relates to the amount of calcium phosphate present in the composition means+/−0.5%. Thus, about 5% means 5+/−0.5%.

The bioactive glass materials may further comprise one or more of a silicate, borosilicate, borate, strontium, or calcium, including SrO, CaO, P₂O₅, SiO₂, and B₂O₃. An exemplary bioactive glass is 45S5, which includes 46.1 mol % SiO₂, 26.9 mol % CaO, 24.4 mol % Na₂O and 2.5 mol % P₂O₅. An exemplary borate bioactive glass is 45S5B1, in which the SiO₂ of 45S5 bioactive glass is replaced by B₂O₃. Other exemplary bioactive glasses include 58S, which includes 60 mol % SiO₂, 36 mol % CaO and 4 mol % P₂O₅, and S70C30, which includes 70 mol % SiO₂ and 30 mol % CaO. In any of these or other bioactive glass materials of the invention, SrO may be substituted for CaO.

The following composition, having a weight % of each element in oxide form in the range indicated, will provide one of several bioactive glass compositions that may be used to form a bioactive glass ceramic:

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-5 |
| CaF | 0-35 |

“Substantially spherical” means 80% of the particles of bioactive glass have an aspect ratio of 1.0+/−0.1 measured as described in more detail below.

Measurements may be taken using a standard light microscope and image analysis software. Biomodal glass are placed on glass slides and viewed using, e.g., a Fisher Scientific Stereomaster microscope with Micron UBS2 software. Digital image processing may be conducted with Image J software from the NIH (Rasband, W S. Image J, Bethesda, Md., USA: National Institutes of Health, 1997-2012). The source images may then be converted to 8-bit images and an ellipse-fit command may be used to approximate the size of the particles. Only those particles that are completely within the analysis frame are measured. The aspect ratio and each particle can be measured automatically by the Image J software.

The bioactive glass ceramic can be in the form of a three-dimensional compressible body of loose glass-based fibers in which the fibers comprise one or more glass-formers selected from the group consisting of P₂O₅, SiO₂, and B₂O₃. The especially small diameter of these fibers renders them highly flexible so they form into the compressible body without breaking. In some embodiments, the body includes fibers meeting these dimensional requirements in addition to other glass morphologies, such as fibers of other dimensions, microspheres, particles, ribbons, flakes or the like. The fibers may have a variety of cross section shapes, such as flat, circular, oval, or non-circular.

Bioactive glasses may be in granular or particulate form, matt or fiber form, a hemostatic sponge, incorporated into a foam, or in the form of a paste or putty. The bioactive glasses may also be in a form of a sphere or a bead, or a combination of all the forms. Exemplary spherical forms were described in U.S. Provisional Application No. 61/786,991, filed Mar. 15, 2013, content of which is incorporated by reference in its entirety. They may also be formulated into settable and non-settable carriers.

A preferred embodiment includes fibers or granules of sol-gel derived bioactive glass in loose form. The bioactive particles, preferably, are comprised of borate based or silicate bioactive glasses and provided in broad, narrow, or blend of particle sizes distribution to control the surface area and interparticle space to achieve specific ion concentrations. In addition, the bioactive glass once placed in contact with the body, interacts with surrounding body fluids to form crystalline hydroxyapatite, which is analogous to bone material.

Bioactive glass ceramics may be prepared by heating a composition comprising one or more of SiO₂, CaH(PO₄), CaO, P₂O, Na₂O, CaCO₃, Na₂CO₃, K₂CO₃, MgO, and H₂BO₃ to a temperature between 1300 and 1500° C. such that the composition may form molten glass. An exemplary composition that can form fibers includes 40-60% SiO₂, 10-20% CaO, 0-4% P₂O₅, and 19-30% NaO. Other exemplary compositions include 45S5, which includes 46.1 mol % SiO₂, 26.9 mol % CaO, 24.4 mol % Na₂O and 2.5 mol % P₂O₅; 45S5B1, which includes 46.1 mol % B₂O₃, 26.9 mol % CaO, 24.4 mol % Na₂O and 2.5 mol % P₂O₅; 58S, which includes 60 mol % SiO₂, 36 mol % CaO and 4 mol % P₂O₅; and S70C30, which includes 70 mol % SiO₂ and 30 mol % CaO. Another exemplary composition includes 40 mol % SiO₂, 40 mol % B₂O₃, 20 mol % CaO, and 20 mol % Na₂O.

Glass particle's size, porosity, and the bonding strength of peptide to glass may be considered when optimizing the releasing characteristics of the peptides. such as fast versus slow rate of release. As a result, different types of peptides can be tailored slow- or fast-releasing depending on the desired timing of the release of the peptide from the glass. For example, small bioactive glass particles may be suitable for use with peptides intended for rapid release, which may be important for early remodeling events. In contrast, larger glass particles may be suitable for use with peptides intended for slow or prolonged release over time that may be important for late remodeling events.

In addition, the size of bioactive glass particles is known to have a large influence on resorption and can confer various additional properties. For instance, small bioactive glass particles, less than 90 microns, can provide for antibacterial activity as well as allowing for rapid release of ions. While small bioactive glass particles may resorb quickly and thus not be available for later stimulation of bone and/or wound healing, the disappearance of these particles may be advantageous by allowing for other cells, such as osteoblasts, to migrate into the bioactive glass particle composition. If larger bioactive glass particles are also present that are slow to resorb, the migrating osteoblasts and other cells can benefit from the ions released by these larger bioactive glass particles. The resulting material having a mixture of bone-forming cells and bioactive glass could serve to promote the formation of more natural bone.

In any embodiment of the invention, the smaller bioactive glass particles may further comprise silver or other metals and agents known to be antibacterial.

In some embodiments of this aspect, the large bioactive glass particles and/or the small bioactive glass particles are microspheres. Glass microspheres are generally known in the art. For instance, U.S. Pat. No. 4,789,501 to Day et al., which is incorporated by reference herein, describes glass microspheres. U.S. Pat. No. 4,904,293 to Garnier et al. and U.S. Pat. No. 5,302,369 to Day et al., which are incorporated by reference herein, also describe glass microspheres. In certain embodiments, it is preferred that the microspheres be substantially spherical and without sharp edges. The microspheres may have an ellipsoidal shape and still be considered substantially spherical.

In some embodiments of this aspect, the large bioactive glass particles have a mean diameter of between about 90 micrometers and about 200 micrometers. In some embodiments, the large bioactive glass particles have a mean diameter of between about 200 micrometers and about 400 micrometers. In some embodiments of this aspect, the large bioactive glass particles have a mean diameter of between about 300 micrometers and about 500 micrometers. In some embodiments of this aspect, the large bioactive glass particles have a mean diameter of between about 400 micrometers and about 600 micrometers. In some embodiments of this aspect, the large bioactive glass particles have a mean diameter of between about 500 micrometers and about 700 micrometers. In some embodiments of this aspect, the large bioactive glass particles have a mean diameter of between about 600 micrometers and about 800 micrometers.

Once the desired bioactive glass composition is prepared, the surface of the bioactive glass may be functionalized by the known methods, including, e.g., plasma-surface modification (PSM), silanation, biotinylation, layer by layer coating assembly, and bioactive glasses' naturally occurring ion exchange mechanism or other available methods.

“Plasma-surface modification” or “PSM” is an effective and economical surface treatment technique for many materials and of growing interests in biomedical engineering. The various common plasma techniques and experimental methods as applied to biomedical materials research, include, e.g., plasma sputtering and etching, plasma implantation, plasma deposition, plasma polymerization, laser plasma deposition, plasma spraying, and so on. The unique advantage of plasma modification is that the surface properties and biocompatibility can be enhanced selectively while the bulk attributes of the materials remain unchanged. Existing materials can, thus, be used and needs for new classes of materials may be obviated thereby shortening the time to develop novel and better biomedical devices.

“Silanation” refers to a process of covering of a surface through self-assembly with organofunctional alkoxysilane molecules. Mineral components like glass surfaces can silanized, because they contain hydroxyl groups which attack and displace the alkoxy groups on the silane thus forming a covalent —Si—O—Si— bond. The goal of silanization is to form bonds across the interface between mineral components and organic components, such as peptides, etc. Silanization (or siliconization) of glass increases its hydrophobicity and reduces adherence of bioglass. Any known silane may be used during the silanation process. Examples of silanes include acrylate and methacrylate, aldehyde, amino, anhydride, azide, carboxylate, phosphonate, sulfonate, epoxy functional, ester, halogen, hydroxyl, isocyanate and masked isocyanate phosphine and phosphate, sulfur, vinyl and olefin, and multi-functional and polymeric silanes. Non-silane coupling agents may also be used. Examples of such agents include zirconates, titanates and aluminates.

“Biotinylation,” in the context of functionalization of the surface of the bioglass, refers to a process of covalently attaching biotin to the surface of the bioglass. Biotinylation is rapid, specific and is unlikely to perturb the natural function of the bioglass due to the small size of biotin (MW=244.31 g/mol). Biotin binds to streptavidin and avidin with an extremely high affinity, fast on-rate, and high specificity, and these interactions are exploited in many areas of biotechnology to isolate biotinylated molecules of interest. Biotin-binding to streptavidin and avidin is resistant to extremes of heat, pH and proteolysis, making capture of biotinylated molecules possible in a wide variety of environments.

“Layer by layer coating assembly” refers to deposition is a thin film fabrication technique. The films are formed by depositing alternating layers of oppositely charged materials with wash steps in between.

Bioactive glasses' naturally occurring “ion exchange mechanism” refers to reversible chemical reaction between two substances (usually a relatively insoluble solid and a solution) during which ions of equal charge may be interchanged.

Once the surface of the bioactive glass is functionalized, the bioactive molecules, peptides, proteins and others can be provided immobilized on the surface of the bioactive glass.

In certain embodiments, the invention relates to any of previously described bioglass compositions provided with surface immobilized peptides.

In certain embodiments, the peptides may be bone resorption inhibitors, such as WP9QY(W9), OP3-4, or RANKL inhibitor peptide and mixtures thereof.

In certain other embodiments, the peptides may be bone formulation stimulators, such as B2A, P1, P2, P3, P4, P24, P15, TP508, OGP, or PTH and mixtures thereof.

In further embodiments, the peptides are both bone resorption inhibitors and bone formation stimulators, such as NBD, CCGRP, or W9 and mixtures thereof.

In yet further embodiments, the peptides may be bone targeting peptides such as (Asp)₆, (Asp)₈, or (Asp, Ser, Ser)₆ and mixtures thereof.

The peptides may be immobilized on the bioglass by any know process, including, e.g., plasma modification, silanation, biotinylation, or layer by layer coating assembly. Exemplary methods of preparing the bioactive glass compositions that include bioglass and surface immobilized peptides are described in detail below.

In further embodiments, provided is a process for making bioactive glasses with surface immobilized peptides.

In certain embodiments, the process may include dissolving one or more peptides, diluting the dissolved one or more peptides, and contacting a bioactive glass with the dissolved peptides to adsorb the one or more peptides on the surface of the bioactive glass, wherein the one or more peptides bind free —OH groups on a surface of the bioactive glass, “Contacting” may be via submerging, coating, spraying or other available methods that provide for absorption of the bioactive molecules, peptides, proteins and others onto the bioactive glass. The step of dissolving one or more peptides may include dissolving one or more peptides in DMSO or another suitable substance. The step of diluting the dissolved one or more peptides may include diluting the dissolved one or more peptides in water or another suitable substance.

In certain other embodiments, the process may include the steps of biotinylating the c-terminus end of one or more peptides, coating a bioactive glass with the one or more biotynilated peptides, blocking the coated bioactive glass, and incubating the blocked bioactive glass for a specified period of time sufficient to allow the attachment of the biotinylated peptides to the bioglass. The blocking step may include contacting the coated bioactive glass with, e.g., serum albumin, polysorbate, EDTA, and sodium nitrate in phosphate buffered saline. The incubating step may include contacting the blocked bioactive glass with, e.g., 4-methylumbelliferyl phosphate substrate in diethanolamine buffer.

The blocking step may be conducted for at least 5 minutes; at least 10 minutes; at least 15 minutes; at least 30 minutes; at least 45 minutes; or at least 1 hour. The blocking step may be conducted for 5 minutes to 1 hour or more; preferably, for 15 minutes to 45 minutes; more preferably 30 minutes to 1 hour; most preferably, 30 minutes. The blocking step may be conducted at temperature from about 20° C. to about 45° C.; more preferably; from about 25° C. to about 35° C.; most preferably at about 30° C. The blocking step may be conducted at temperature of at least, 20° C.; more preferably, at least, 25° C.; most preferably, at least, 30° C. or higher.

Certain other embodiments, relate to a method for making bioactive glass coated with surface immobilized peptides that includes comprising silanating one or more peptides, coating a bioactive glass with the one or more biotynilated peptides, blocking the coated bioactive glass, and incubating the blocked bioactive glass. The silanating step includes contacting the one or more peptides with 4-aminobutyltriethoxysilane.

Yet another aspect of the invention provides for a putty composition, which comprises bioactive glass with surface immobilized peptides and further containing glycerin, and polyethylene glycol. The bioactive glass composition is comprised of large bioactive glass particles and small bioactive glass particles. The large bioactive glass particles have a substantially spherical shape and a mean diameter of between about 90 micrometers and about 2000 micrometers. The small bioactive glass particles have a substantially spherical shape and a mean diameter of between about 10 micrometers and about 500 micrometers.

The putty composition, which includes all compositions having a putty-like composition, has the advantages of being moldable and adhesive at room temperature. An ideal putty composition does not swell in the presence of biological fluids and does not dry out rapidly. The putty may be applied to gaps in the bone or the skeletal system, along with other bony defects.

The putty composition may be effective to fill bone defects, gaps in bone, and gaps in the skeletal system. The putty composition may also be effective for dental bony defects. Such defects and gaps may be surgically-created or arise from traumatic injury to the bone. The glycerin and polyethylene glycol of the putty may serve as a carrier for the bioactive glass with surface immobilized peptides. The putty may be applied manually at the site or near the site of bone defects, gaps in bone, and gaps in the skeletal system.

Bioactive glass is capable of bonding to bone, which begins with the exposure of bioactive glass to aqueous solutions. Sodium ions in the glass can exchange with hydronium ions in body fluids, which increases the pH. Calcium and phosphorous ions can migrate from the glass to form a calcium and phosphate-rich surface layer. Borate ions can also migrate from the glass to from a surface layer rich in boron. Strontium ions also can migrate from the glass to form a strontium-rich surface layer. Underlying this surface layer is another layer that becomes increasingly silica rich due to the loss of sodium, calcium, strontium, boron, and/or phosphate ions (U.S. Pat. No. 4,851,046). Hydrolysis may then disrupt the Si—O—Si bridges in the silica layer to form silanol groups, which can disrupt the glass network. The glass network is then thought to form a gel in which calcium phosphate from the surface layer accumulates. Mineralization may then occur as calcium phosphate becomes crystalline hydroxyapatite, which effectively mimics the mineral layer of bones.

In certain embodiments, application of bioactive glass to bone may promote bone remodeling. Bone remodeling occurs by equilibrium between osteoblast-mediated bone formation and osteoclast-mediated bone destruction. When bone is injured or missing, such as in a fracture, promotion of osteoblast activity is thought to be helpful to induce bone formation. Further, promoting bone formation by osteoblasts may be helpful in locations in which there is significant bone loss in the absence of an apparent injury. As such, certain embodiments relate to a method for promoting bone remodeling in a subject. The method includes contacting the bone in need of bone remodeling with any of the bioactive glass composition with surface immobilized molecules, peptides, or the like.

The bioactive glass may have osteostimulative properties, which refers to promoting proliferation of the osteoblasts, such that bone can regenerate. In an osteostimulative process, a bioactive glass material may be colonized by osteogenic stem cells. This may lead to quicker filling of bone defects than would otherwise occur with an osteoconductive glass.

In various embodiments of this and other aspects of the invention, the bioactive glass sheets, fibers, and mesh may provide structure to a tissue site in order to support, promote or facilitate new tissue growth.

In certain embodiments, the bonding of bioactive glass to bone begins with the exposure of bioactive glass to aqueous solutions. Sodium ions in the glass can exchange with hydronium ions in body fluids, which increases the pH. Calcium and phosphorous ions can migrate from the glass to form a calcium and phosphate-rich surface layer. Borate ions can also migrate from the glass to from a surface layer rich in boron. Strontium ions also can migrate from the glass to form a strontium-rich surface layer. Underlying this surface layer of the bioactive glass is another layer, which becomes increasingly silica-rich due to the loss of sodium, calcium, strontium, boron, and/or phosphate ions (U.S. Pat. No. 4,851,046). Hydrolysis may then disrupt the Si—O—Si bridges in the silica layer to form silanol groups, which can disrupt the glass network. The glass network is then thought to form a gel in which calcium phosphate from the surface layer accumulates. Mineralization may then occur as calcium phosphate becomes crystalline hydroxyapatite, which effectively mimics the mineral layer of bones.

In certain embodiments, the bioactive glass compositions may be used for filling bone defects, gaps in bone or gaps in skeletal system of a subject. The bioactive glass composition, when placed in contact with a bone at or near a site of a bone defect, is capable of eliminating the adsorption of proteins that would result in the adhesion of unspecific cells leading to fibrous integration, enhancing the specific attachment of osteogenic cells for the establishment of a tight bone-implant interface; and providing integrin-mediated signals for provoking bone healing mechanisms.

As such, certain embodiments relate to a method of treating a bone having a bone defect comprising contacting the bone at or near the site of the bone defect with the bioactive glass compositions described herein.

A further aspect of the invention provides for a method for treating a bone having a bone defect comprising contacting the bone at or near the site of the bone defect with any of the previously-described bimodal bioactive glass compositions.

It is also within the scope of the present invention to combine any of the previously-described bioactive glass with surface immobilized peptides with other wound and bone repair treatments such as antimicrobials, antibiotics, collagen, fibrin, fibronectin, vitamin E, other wound or bone repair dressings/treatments known to those of ordinary skill in the art.

Any of the above-described aspects of the invention may be used in any number of applications. One such application involves spinal and orthopedic procedures. For example, a material of the invention can be surgically placed near bone voids or worn portions of bones, such as discs.

Certain embodiments relate to a method for treating a bone having a bone defect comprising contacting the bone at or near the site of the bone defect with any of the above-described bioactive glasses with surface immobilized peptides.

Certain other embodiments relate to an implant with a coating of bioactive glass with surface immobilized peptides.

Another application involves bone repair and restoration. The inventive materials may be used in conjunction with other orthopedic devices such as joints, pins, rods, anchors, rivets, staples, screws, etc. Other pastes, cements, and fluids used in orthopedic restoration may be combined with any of the inventive materials. Meshes may also be used in conjunction with any of the materials described herein to promote repair in a load-bearing application.

Yet another application involves dental procedures, such as bone grafting. For example, the inventive materials may be used to reduce bone loss at or near sites of oral surgery.

### Example 1

Peptides were added with concentration of 0.02% mole peptide per mole of SiO₂ as calculated present in each of the specimens. RODI-H₂O was used specifically to limit the formation of the HA layer formation until the added peptides adsorb to the surface of the materials. The desired amount of each peptide was dissolved in 50 μl of dimethyl sulfoxide (DMSO) (Sigma) then further diluted in autoclaved DI-H₂O and mixed so that the required concentration and combination of peptides was obtained. The peptide solutions were then added to the samples and left for 24 hour to allow the peptides to bond to free —OH groups on the surface of the bioactive glass before adding culture media.

### Example 2

The carboxyl end of the peptide was biotinylated using an EZ-Link_{—} Amine-PEG3-Biotin kit (Pierce Biotechnology), according to manufacturer's instructions. Unreacted biotin was removed via overnight dialysis against PBS using a Slide-A-Lyzer Dialysis Cassette with a molecular weight cut-off of 3500 (Thermo Scientific). After dialysis, protein concentration was measured using a BCA Protein Assay kit (Pierce Biotechnology).

Bioactive glass was coated with various concentrations of biotinylated peptide, then blocked in 0.25% heat denatured serum albumin with 0.0005% Tween-20, 1 mM EDTA, and 0.025% NaN₃ in PBS for 1 h at 37° C. After blocking, scaffolds were incubated with an anti-biotin antibody conjugated to alkaline phosphatase (clone BN-34, Sigma, 1:2000) for 1 h at 37° C., then incubated with 60 mg/mL of 4-methylumbelliferyl phosphate substrate in diethanolamine buffer (pH 9.5) for 1 h at 37° C. Fluorescent signal was detected on an HTS 7000 Plus Bio Assay Reader (Perkin Elmer) at 360/465 nm (ex/em) by transferring 100 mL of reaction solution from each scaffold to a microtiter plate. Uncoated bioactive glass scaffolds and a substrate-only group (excluding the antibody) served as negative controls.

### Example 3

### Surface Functionalization of Bioactive Glass by Silanation with 4-Aminobutyltriethoxysilane and Subsequent Surface Immobilization with Peptides

Protocol for Preparing Bioactive Glass:

Weigh 100 g of 1-2 mm calcium phosphate into a mixing bowl. Prepare the silane solution from the materials listed in the top half of the chart below and pour the solution into a spray bottle. Weigh the spray bottle containing the solution and record the weight. Spray-apply the silane solution to the calcium phosphate while continually mixing the TCP. After 2-3 sprays, weigh the spray bottle and record the change in weight. Continue to apply the silane solution until the change in weight is equivalent to the weight of silane solution listed in the table above (i.e.: 7.83 g of solution for 1% silicated BG). After the TEOS solution has been applied, continue mixing BG for 5-10 minutes, occasionally scraping the walls and bottom of bowl. Place a lid on the mixing bowl to and incubate the treated calcium phosphate in an oven for 120 hours at 50° C. Following incubation, pour the treated bioactive glass onto a drying tray and place the TCP back into oven at 50° C. Dry the bioactive glass for 1 week at 50° C. to burn off residual ethanol and acetic acid.

| | | | | |
|---|---|---|---|---|
| Material | % | MW | 25 g | 50 |

4-aminobutyltriethoxysilane Solution

| | | | | |
|---|---|---|---|---|
| Silane | 50.00 | 235.4 | 12.5 | 25.00 |
| Ethyl Alcohol | 40.00 | 46.00 | 10 | 20.00 |
| Acetic Acid | 5.00 | 74.00 | 1.25 | 2.50 |
| Water | 5.00 | 18.00 | 1.25 | 2.50 |

Silicated BG Formulations

| | | | | |
|---|---|---|---|---|
| wt % Coating | 0.1% | 1% | 3% | 5% |
| Calcium | 100.00 | 100.00 | 100.00 | 100.00 |
| Phosphate (g) | | | | |
| Solution (g) | 0.78 | 7.83 | 23.50 | 39.17 |

Once the bioglass is prepared, peptides are added with concentration of 0.02% mole peptide per mole of SiO₂ as calculated present in each of the specimens. RODI-H₂O is used specifically to limit the formation of the HA layer formation until the added peptides adsorb to the surface of the materials. The desired amount of each peptide is dissolved in 50 μl of dimethyl sulfoxide (DMSO) (Sigma) then further diluted in autoclaved DI-H₂O and mixed so that the required concentration and combination of peptides is obtained. The peptide solutions are then added to the samples and left for 24 hour to allow the peptides to bond to free —OH groups on the surface of the bioactive glass before adding culture media.

Throughout this specification various indications have been given as preferred and alternative embodiments of the invention. However, the foregoing detailed description is to be regarded as illustrative rather than limiting and the invention is not limited to any one of the preferred embodiments. It should be understood that it is the appended claims, including all equivalents that are intended to define the spirit and scope of this invention.

1. A bioactive glass composition comprising bioactive glass with surface immobilized peptides, wherein the peptides are selected from WP9QY(W9), OP3-4, or RANKL inhibitor peptide, and mixtures thereof.

2. A bioactive glass composition comprising bioactive glass with surface immobilized peptides, wherein the peptides are selected from B2A, P1, P2, P3, P4, P24, P15, TP508, OGP, or PTH, and mixtures thereof.

3. A bioactive glass composition comprising bioactive glass with surface immobilized peptides, wherein the peptides are selected from NBD, CCGRP, or W9, and mixtures thereof.

4. A bioactive glass composition comprising bioactive glass with surface immobilized peptides, wherein the peptides are selected from (Asp)₆, (Asp)₈, or (Asp, Ser, Ser)₆, and mixtures thereof.

5. A bioactive glass composition comprising bioactive glass with surface immobilized peptides, wherein the peptides are selected from WP9QY(W9), OP3-4, RANKL, B2A, P1, P2, P3, P4, P24, P15, TP508, OGP, PTH, NBD, CCGRP, W9, (Asp)₆, (Asp)₈, or (Asp, Ser, Ser)₆, and mixtures thereof.

6. The bioactive glass composition of any of claims 1-5, wherein the bioglass is in a granular form, particulate form, matt form, fiber form, hemostatic sponge form, foam form, paste or putty form, or sphere or bead form, or a combination thereof.

7. The bioactive glass composition of any of claims 1-5, wherein the bioglass is selected from the group consisting of 45S5 bioglass, 45S5B1, 58S, S70C30, and mixtures thereof.

8. The bioactive glass composition of any of claims 1-5, wherein the bioglass is porous.

9. The bioactive glass composition of any of claims 1-5, further comprising at least one therapeutic agent selected from the group consisting of antimicrobials, antibiotics, collagen, fibrin, fibronectin, Vitamin E, would repair dressing, and mixtures thereof.

10. The bioactive glass composition of any of claims 1-5, wherein the composition is for filling bone defects, gaps in bone or gaps in skeletal system of a subject.

11. The bioactive glass composition of any of claims 1-5, wherein the composition, when placed in contact with a bone at or near a site of a bone defect, is capable of eliminating the adsorption of proteins that would result in the adhesion of unspecific cells leading to fibrous integration, enhancing the specific attachment of osteogenic cells for the establishment of a tight bone-implant interface, and providing integrin-mediated signals for provoking bone healing mechanisms.

12. The bioactive glass composition of any of claims 1-5, wherein the peptides are immobilized on the bioglass by process of plasma modification, silanation, biotinylation, or layer by layer coating assembly.

13. A method of treating a bone having a bone defect comprising contacting the bone at or near the site of the bone defect with the bioactive glass composition of any of claims 1-5.

14. A method for making bioactive glass coated with surface immobilized peptides comprising:
a. dissolving one or more peptides,
b. diluting the dissolved one or more peptides, and;
c. contacting a bioactive glass with the dissolved peptides to adsorb the one or more peptides on the surface of the bioactive glass, wherein the one or more peptides bind free —OH groups on a surface of the bioactive glass.

15. A method for making bioactive glass coated with surface immobilized peptides comprising:
a. biotinylating the c-terminus end of one or more peptides,
b. coating a bioactive glass with the one or more biotynilated peptides,
c. blocking the coated bioactive glass, and;
d. incubating the blocked bioactive glass.

16. The method of claim 15, wherein the blocking step comprises contacting the coated bioactive glass with serum albumin, polysorbate, EDTA, or sodium nitrate in phosphate buffered saline.

17. The method of any of claims 15-16, wherein the incubating step comprises contacting the blocked bioactive glass with 4-methylumbelliferyl phosphate substrate in diethanolamine buffer.

18. A method for making bioactive glass coated with surface immobilized peptides comprising:
a. silanating one or more peptides,
b. coating a bioactive glass with the one or more biotynilated peptides,
c. blocking the coated bioactive glass, and;
d. incubating the blocked bioactive glass.

19. The method of claim 18, wherein the silanating step comprises contacting the one or more peptides with 4-aminobutyltriethoxysilane.

20. The method of claim 14, wherein the peptides are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

21. A method for promoting bone remodeling in a subject comprising contacting the bone in need of bone remodeling with the bioactive glass composition of any of claims 1-5.

22. The method of claim 15, wherein the peptides are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

23. The method of claim 16, wherein the peptides are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

24. The method of claim 17, wherein the peptides are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

25. The method of claim 18, wherein the peptides are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

26. The method claim 19, wherein the peptides are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.
